(19) United States
(12) Patent Application Publication
(10) Pub. No.: US 2019/0231609 A1
(43) Pub. Date: Aug. 1, 2019

(54) WOMAN SANITARY FLUID CAPTURE SYSTEM

(71) Applicant: Gokulakrishnan Sankar, Arlington, TX (US)

(72) Inventor: Gokulakrishnan Sankar, Arlington, TX (US)

(21) Appl. No.: 16/261,453

(22) Filed: Jan. 29, 2019

Related U.S. Application Data
(60) Provisional application No. 62/623,427, filed on Jan. 29, 2018.

(51) Int. Cl. A61F 13/472

(52) U.S. Cl. CPC A61F 13/47236; A61F 2013/53765

(57) ABSTRACT

A sanitary fluid capture system allows for better fluid transfer to an absorbent pad. The system places moisture capturing and transferring materials in contact with the source of the fluids. Absorbent material forms a basin to capture excess fluid flow while it is sequestered by the absorbent materials.

### BACKGROUND

### 1. Field of the Invention

The present invention relates generally to feminine hygiene systems, and more specifically, to a devices and methods for capturing fluid discharge.

### 2. Description of Related Art

Feminine hygiene systems are well known in the art and are effective means to care for needs particular to the female body. For example, FIG. 1 depicts a conventional absorbent hygiene pad 101 having an absorbent layer 103 surrounded by a permeable cover 105. During use, the pad 101 is positioned near an orifice to collect fluid 107 in the absorbent layer 103.

One of the problems commonly associated with absorbent hygiene pad 101 is its limited efficiency. For example, when fluid exits the orifice it is able to flow in multiple directions prior to contact with the pad allowing for leaks, spills or the like. Additionally, the pad does not conform to the complex geometry of the body, further increases chances for spills.

Accordingly, although great strides have been made in the area of feminine hygiene systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a top view of a common absorbent hygiene pad;

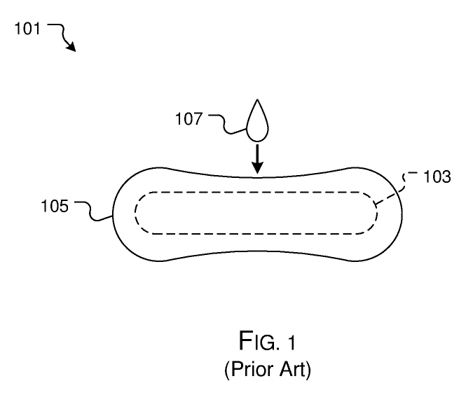

FIG. 2 is a cross-sectional front view of a sanitary fluid capture system in accordance with a preferred embodiment of the present application; and

FIG. 3 is a top view of an alternative embodiment of the system of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional absorbent hygiene pad. Specifically, the invention of the present application prevents spills or leaks by conforming to the surface of the body and providing a direct path for the fluid to follow to the absorbent portion of the invention. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

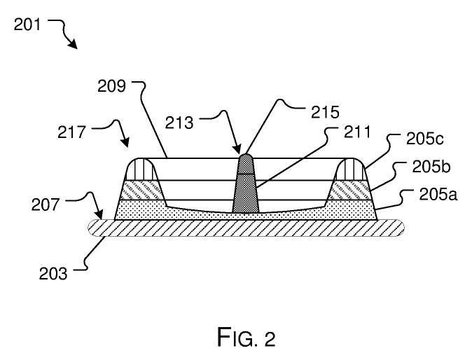

Referring now to the drawings wherein like reference characters identify corresponding or similar elements throughout the several views, FIG. 2 depicts a cross-sectional front view of a sanitary fluid capture system in accordance with a preferred embodiment of the present application. It will be appreciated that system 201 overcomes one or more of the above-listed problems commonly associated with conventional absorbent hygiene pads.

In the contemplated embodiment, system 201 includes a base layer 203 of non-permeable material having a stack 217 of absorbent layers 205 attached to the upper surface 207 thereof. The absorbent layers 205 are of decreasing size and configured to form a basin 209 within the stack. In the center of the basin 209 an absorbent body 211 extends away from the upper surface 207. A first end 213 thereof having a wicking portion 215.

In use, the system 201 is placed near an orifice from which fluid is discharged, the wicking portion 215 being placed in fluid communication with the orifice. The fluid travels through the wicking portion 215 to the absorbent layers 205. If a large volume of fluid is discharged it is captured in the basin 209 till it enters the absorbent layers 205.

It should be appreciated that one of the unique features believed characteristic of the present application is that basin 209 and absorbent body 211 rise from the upper surface 207 to contact or substantially close any gaps between the system and the geometry of the area around the orifice.

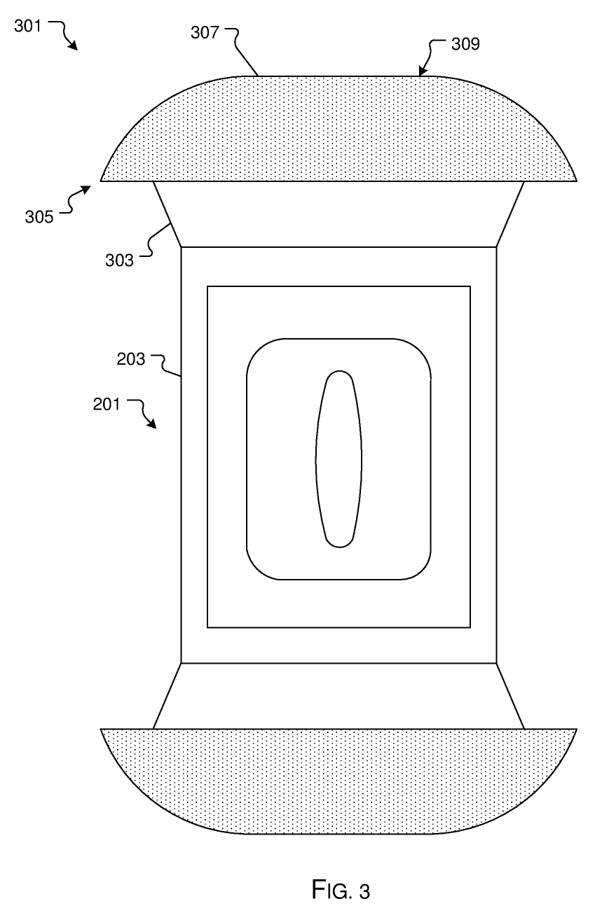

Referring now to FIG. 3 an alternative embodiment of the system 201 is depicted. Embodiment 301 including a supportive layer 303 attached below the base layer 203. The supportive layer 303 having arms 305 extending away from the base layer 203 having adhesive 307 attached to the tips 309 or edge thereof.

It will be appreciated that the arms 305 and adhesive 307 enable the embodiment 301 to be attached to skin of a user so that it remains in position when attached.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. A sanitary fluid capture system comprising:
at least one base layer having at least two absorbent layers attached to the top surface thereof; and
the absorbent layers forming a stack so that a basin is formed;
an absorbent body attached in center of the basin and extending up and away from the upper surface;
the absorbent body having a wicking portion at the distal end;
wherein the top of the stack of the absorbent layers and the wicking portion are in proximity to an orifice; and
wherein fluid from the orifice travels through the wicking portion to the absorbent body where it is captured.

2. The system of claim 1 wherein the base layer is attached to a supportive layer having adhesive attached to the tips of arms extending away from the base layer.
